(19) United States
(12) Patent Application Publication
(10) Pub. No.: US 2021/0326995 A1
(43) Pub. Date: Oct. 21, 2021

(54) CLAIM SETTLEMENT ANTI-FRAUD METHOD, APPARATUS, DEVICE, AND STORAGE MEDIUM BASED ON GRAPH COMPUTATION TECHNOLOGY

(71) Applicant: PING AN TECHNOLOGY (SHENZHEN) CO., LTD., Shenzhen, Guangdong (CN)

(72) Inventors: Jianzong Wang, Shenzhen, Guangdong (CN); Zhangcheng Huang, Shenzhen, Guangdong (CN)

(21) Appl. No.: 17/263,899

(22) PCT Filed: Nov. 12, 2019
(86) PCT No.: PCT/CN2019/117708

§ 371 Date: Jan. 28, 2021

(30) Foreign Application Priority Data
Jan. 23, 2019 (CN) 201910064223.7

(51) Int. Cl. G06Q 40/08; G06Q 20/40; G16H 10/60; G16H 40/20; G16H 50/70; G16H 50/20; G06F 16/901; G06F 16/906

(52) U.S. Cl. CPC G06Q 40/08; G06Q 20/4016; G16H 10/60; G06F 16/906; G16H 50/70; G16H 50/20; G06F 16/9024; G16H 40/20

(57) ABSTRACT

A claim settlement anti-fraud method, an apparatus, a computer device, and a storage medium are provided. The method includes generating a sub-graph of doctor and patient, a sub-graph of doctor and medical advice, and a fused large graph according to medical data. A patient relationship network with several community close loops is generated by mapping the sub-graph of doctor and patient according to the fuses large graph. A similarity between any two vertexes in the patient relationship network are computed. An average similarity of each community close loop is computed. The insurance fraud actions are confirmed based on the average similarity.

### CROSS-REFERENCE TO RELATED APPLICATION

The present application claims priority to Chinese Patent Application No. 201910064223.7 entitled “CLAIM SETTLEMENT ANTI-FRAUD METHOD, APPARATUS, DEVICE, AND STORAGE MEDIUM BASED ON GRAPH COMPUTATION TECHNOLOGY” filed on Jan. 23, 2019, the contents of which is expressly incorporated by reference herein in its entirety.

### TECHNICAL FIELD

The present disclosure relates an Internet financial field, particular to a claim settlement anti-fraud method, an apparatus, a device, and a storage medium based on graph computation.

### BACKGROUND

Data in a financial social security field is huge and complex. By comparing with traditional database technology, a graph computation technology effectively mine the interdependent valuations between data. In graph data, a vertex normally represents an entity object, and an edge represents a relation between entity objects, different types of graph data are established based on different application scenarios, which are capable of establishing complex reality network and model, and completely reflect reality problems. The financial social security needs to be better maintained, a financial system needs to be protected, violations need to be cracked down, especially patient group insurance frauds actions. However, insurance frauds behaviors are normally confirmed by artificial investigations, and it is hard to quickly and correctly confirm the insurance frauds behaviors, and it is laborious and time consuming. Thus, it is necessary to provide a claim settlement anti-fraud method based on graph computation for screening the insurance frauds actions of patient groups.

### SUMMARY OF THE INVENTION

The present disclosure provides a claim settlement anti-fraud method based on graph computation technology, apparatus, device, and a storage medium, aiming to provide an important reference for screening the insurance frauds actions.

Firstly, the present disclosure provides a claim settlement anti-fraud method based on graph computation technology, the method includes:

Generating a sub-graph of doctor and patient and a sub-graph of doctor and medical advice according to medical data, and generating a fused large graph according to the sub-graph of doctor and patient and the sub-graph of doctor and medical advice, based on a graph computation technology;

Generating a patient relationship network by mapping the sub-graph of doctor and patient according to the fused large graph; the patient relationship network includes a plurality of community close loops;

Computing a similarity between any two vertexes in the patient relationship network according to feature parameters of patients corresponding to the any two vertexes in the patient relationship network;

Computing an average similarity of each community close loop according to the similarity; and

Confirming insurance fraud actions according to the average similarity.

Secondly, the present disclosure provides a claim settlement anti-fraud apparatus based on graph computation technology, the claim settlement anti-fraud apparatus includes:

A graph generation module, configured to generate a sub-graph of doctor and patient and a sub-graph of doctor and medical advice according to medical data, and generate a fused graph according to the sub-graph of doctor and patient and the sub-graph of doctor and medical advice, based on a graph computation technology;

A network generation module, configured to generate a patient relationship network by mapping the sub-graph of doctor and patient according to the blended graph; the patient relationship network includes a plurality of community close loops;

A first computation module, configured to compute a similarity between any two vertexes in the patient relationship network according to feature parameters of patients corresponding to the any two vertexes in the patient relationship network;

A second computation module, configured to compute an average similarity of each community close loop according to the similarity; and

An insurance fraud confirming module, configured to confirm insurance fraud actions according to the average similarity.

Thirdly, the present disclosure provides a computer device. The computer device includes a storage and a processor. The storage stores computer programs. The processor executes the computer programs to implement the above claim settlement anti-fraud method.

Fourthly, the present disclosure provides a computer readable medium. The computer readable medium stores computer programs. The computer programs are executed by a processor for implementing the above claim settlement anti-fraud method.

The present disclosure provides a claim settlement anti-fraud method based on graph computation technology, an apparatus, a device, and a storage medium. The sub-graph of doctor and patient, the sub-graph of doctor and medical advice, and the fused large graph are generated according to the medical data. A patient relationship network is generated by mapping the sub-graph of doctor and patient according to the fused graph. The patient relationship network includes a plurality of community close loops. The similarity between any two vertexes in the patient relationship network are computed according to feature parameters of patients corresponding to the any two vertexes in the patient relationship network. The average similarity of each community close loop is computed according to the similarity. The insurance fraud actions are confirmed according to the average similarity.

### BRIEF DESCRIPTION OF THE FIGURES

Implementations of the present disclosure will now be described, by way of example only, with reference to the attached figures, wherein:

FIG. 1 is a flowchart of an embodiment of a method for establishing graph data based on graph computation technology.

FIG. 2 is a diagram view of an embodiment of a sub-graph of doctor and patient.

FIG. 3 is a diagram view of an embodiment of a sub-graph of doctor and medical advice.

FIG. 4a is a principle schematic diagram view of a first embodiment of a large graph fused by the sub-graphs.

FIG. 4b is a structure diagram view of a second embodiment of the fused large graph.

FIG. 5 is a flowchart of an embodiment of a claim settlement anti-fraud method based on graph computation technology.

FIG. 6 is a detailed flowchart of sub-steps in the claim settlement anti-fraud method of FIG. 5.

FIG. 7 is a diagram view of an embodiment of the patient relationship network.

FIG. 8 is detailed flowchart of sub-steps of the claim settlement anti-fraud method of FIG. 5.

FIG. 9 is a diagram view of an embodiment of a doctor clustering result.

FIG. 10 is a diagram view of an embodiment of the patient relationship network after clustering.

FIG. 11 is a diagram view of an embodiment of the patient relationship network after updating weight.

FIG. 12 is a diagram view of a second embodiment of a claim settlement anti-fraud method.

FIG. 13 is a detail diagram view of sub-steps of the claim settlement anti-fraud method of FIG. 12.

FIG. 14 is a diagram view of an embodiment of a doctor relationship network.

FIG. 15 is a detail diagram view of sub-steps of the claim settlement anti-fraud method of FIG. 12.

FIG. 16 is a diagram view of a first embodiment of a claim settlement anti-fraud apparatus.

FIG. 17 is a diagram view of a second embodiment of a claim settlement anti-fraud apparatus.

FIG. 18 is a diagram view of an embodiment of a computer device.

### DETAILED DESCRIPTION

In order to making the technical solutions of the present disclose to be clearer and more understand, the present disclosure is described in detail with reference to the accompanying drawings and the embodiments. Obviously, the specific embodiments described herein are several embodiments of the present disclosure, but not the entire embodiments of the present disclosure. Other embodiment achieved according to the specific embodiments described herein by those of an ordinary skill in the art are within the protecting range of the present disclosure.

The flowchart in drawings is merely an example, the content and operations or steps are not necessary, nor being implemented as the described sequence. For example, some operations/steps can be decomposed, combined, or partially combined, and the execution sequence can be change due to action conditions.

The present disclosure provides a claim settlement anti-fraud method based on a graph computation technology, an apparatus, a computer device, and a storage medium. The claim settlement anti-fraud method based on the graph computation technology provides an important reference for quickly identifying insurances fraud of patients and/or doctors.

Hereinafter, some embodiment of the present disclose are further described in detail with reference to the accompanying drawings and the embodiments. The embodiments and features in the embodiments can be combined with each other without conflict.

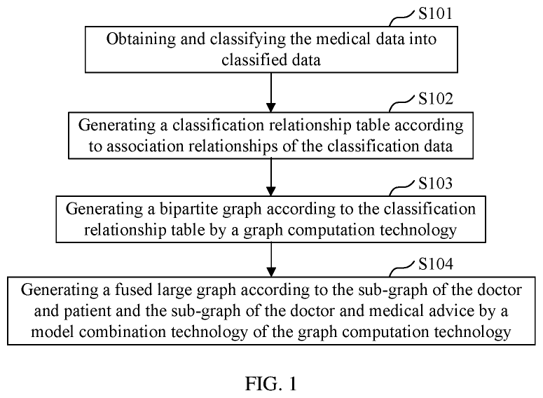

As shown in FIG. 1, a flowchart of a method for establishing a graph data based on the graph computation technology. The method for establishing the graph data strips entities and relationships of structural data stored in a traditional database. The entities and the relationships are mapped into vertexes and edges. The vertexes and the edges are converted into graph data to be stored in the network. The traditional database can be Structured Query Language (SQL).

As shown in FIG. 1, the steps of establishing the graph data based on the graph computation technology is configured to establish medical graph data according to medical data for achieving claim settlement anti-fraud according to the medical data. The steps of establishing the graph data based on the graph computation technology includes:

In block S101, obtaining and classifying the medical data into classified data.

In one embodiment of the present disclose, the classified data includes patient basic information, doctor basic information, and medical advice information. Certainly, there can be other information types in the classified data.

In detail, the patient basic information, doctor basic information, and medical advice information are shown in tabular form, which are a patient basic information table, a doctor basic information table, and a medical advice information table.

The patient basic information table includes patient serial number, sex, age, and health insurance number. The doctor basic information table includes doctor serial number and department number. The medical advice information table includes medical advice, unit price of the medical advice, and subclass of the medical advice. In detail, the tables are shown as below.

TABLE 1
Patient basic information table

| | Patient | | | Live or | Health insurance |
|---|---|---|---|---|---|
| ID1 | number | Gender | Age | dead | number |
| 1 | 34323243 | 1 | 23 | 0 | 655231 |
| 2 | 34323244 | 2 | 32 | 0 | 655321 |
| 3 | 34323245 | 1 | 78 | 1 | 655767 |
| 4 | 34323246 | 2 | 44 | 0 | 1 |
| 5 | . . . | . . . | . . . | . . . | . . . |

In table 1, in ID column, Arabic numeral or letters represent unique identifications of different patients. In the patient number column, a string of number as the patient serial number represents different patients. In sex column, 1 represents male, and 2 represents female. In live or dead column, 0 represents alive, and 1 represents death. In health insurance column, 1 represents a lack of health insurance card, and otherwise a string number represents a number of the health insurance card.

TABLE 2
Doctor basic information table

| ID2 | Doctor number | Department |
|---|---|---|
| 1 | 1001 | 201 |
| 2 | 1002 | 201 |
| 3 | 1003 | 202 |
| 4 | 1004 | 203 |
| 5 | . . . | . . . |

In the table 2, in ID2 column, Arabic numeral or letters represent unique identifications of different doctors. In doctor number column, a string of number as the doctor serial number represents different doctors. In department column, the department serial number represents the department which the doctor belongs to.

TABLE 3
Medical advice table

| | Medical advice | | Medical advice |
|---|---|---|---|
| ID3 | item | Price | category |
| 1 | 18275∥1 | 0.77 | 6 |
| 2 | 10292∥1 | 1.6 | 6 |
| 3 | 19022∥1 | 2.6 | 1 |
| 4 | 12733∥1 | 6.6 | 2 |
| 5 | . . . | . . . | |

In table 3, in ID3 column, Arabic numeral or letters represent unique identifications of different medical advices. In medical advice item, a string of number as the medical advice represents different medical advice contents. In price column, the number represents an amount cost, the unit is RMB. In medical advice category, serial number represents different categories of the medical advice information.

It needs to explain that, all the patient serial number, the doctor serial number, the department number, and the medical advices can be numbered in different manner according to different hospitals or different medical institutions.

In block S102, generating a classification relationship table according to association relationships of the classification data.

The association relationship is generated by extracting association information from the patient basic information, doctor basic information, and medical information. The association relationship is a connection between the classification data. The classification relationship table is generated according to the association of the classification data. The classification relationship table includes a patient and doctor relationship table and a doctor and medical advice relationship table.

The association relationship is a connection between the classification data. For example, the patient sees a doctor to form a patient and doctor association relationship. The doctor gives a medical advice to forma a doctor and medical advice association relationship. Two different patients went to a same hospital or saw a same doctor, or two different doctors saw a same patient. The above connections are all association relationship.

In detail, the association relationship extracted from the patient basic information, the doctor basic information, and medical advice information are graphed as the classification relationship table. In one embodiment, the classification relationship table includes a patient and doctor relationship table and a doctor and medical advice relationship table.

The patient and doctor relationship table are shown as Table 4.

TABLE 4
Patient and doctor relationship table

| ID1 | ID2 | Visiting data | Fee category | Bill number |
|---|---|---|---|---|
| 1 | 4 | 2014-1-1 | 3 | 541212 |
| 2 | 1 | 2014-1-1 | 2 | 541243 |
| 3 | 1 | 2014-1-2 | 2 | 541266 |
| 4 | 2 | 2014-1-3 | 1 | 541276 |
| 5 | . . . | . . . | | |

In table 4, in ID1 column represents a patient identification, and ID2 column represents a doctor identification. In visiting data column represents a data when the patient sees the doctor. In fee category column, 1 represents self-pay manner, 2 and 3 represent health insurance reimbursement manner. In bill number column, a bill serial number represents the bill of different patients.

TABLE 5
Doctor and medical advice relationship table

| ID2 | ID3 | Advice time | Amount | Bill number |
|---|---|---|---|---|
| 1 | 3 | 7:59:45 | 32 | 543211 |
| 2 | 1 | 7:59:45 | 12 | 546533 |
| 4 | 2 | 7:59:55 | 9 | 541433 |
| 4 | 6 | 7:56:44 | 33 | 541565 |
| 5 | . . . | . . . | | |

In table 5, ID2 column represents a doctor, and ID3 column represents a medical advice. The advice time represents a time of the medical advice of the patient provided by the doctor. In amount column, the number represents a number of the medical advices. In bill number column, the bill serial number represents bills of different patients.

In block S103, generating a bipartite graph according to the classification relationship table by a graph computation technology.

The bipartite graph includes a sub-graph of doctor and patient and a sub-graph of doctor and medical advice. In detail, the sub-graph of doctor and patient are generated according to the patient and doctor relationship table by the graph computation technology, and the sub-graph of doctor and medical advice are generated according to the doctor and medical advice relationship table by the graph computation technology.

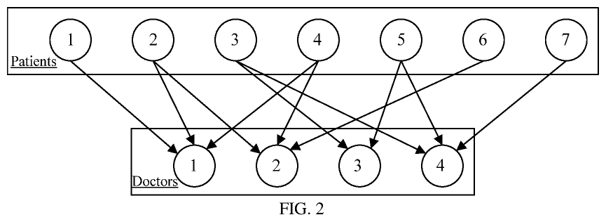

The sub-graph of doctor and patient are shown in FIG. 2. The patients and doctors are served as vertexes by using patient ID1 and doctor ID2 as labels. The department number of the doctor are served as properties of the vertexes. Other information of the patient is served as edge properties, such as the visiting time, the fee category.

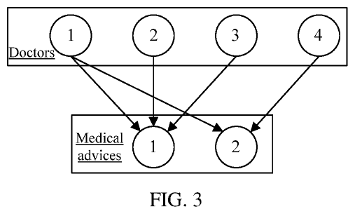

The sub-graph of doctor and medical advice are shown in FIG. 3. The doctors and medical advices are served as vertexes by using doctor ID2 and medical advice ID3. The department number of the doctor are served as properties of the vertexes. Other information of the medical advice is served as edge properties, such as the advising time, the amount of the medical advice.

In block S104, generating a fused large graph according to the sub-graph of doctor and patient and the sub-graph of doctor and medical advice by a model combination technology of the graph computation technology.

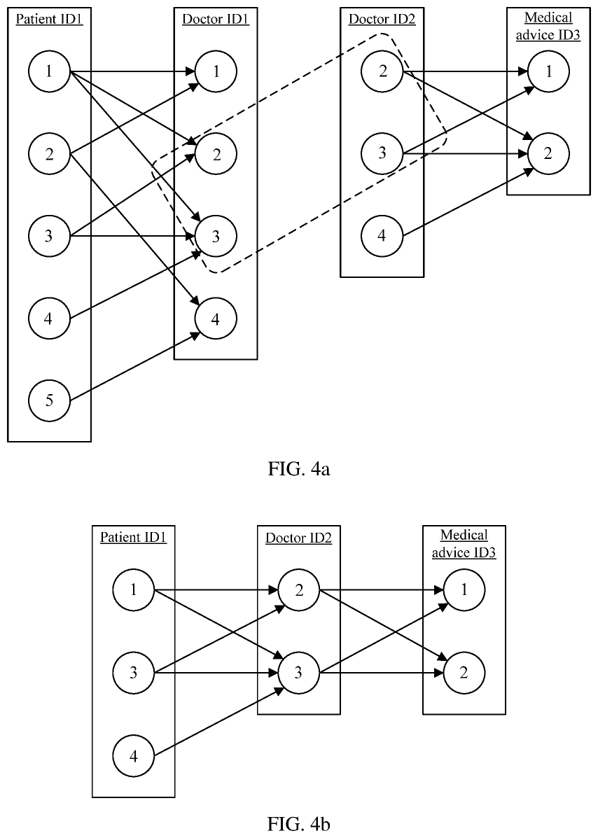

In detail, as shown in FIGS. 4a and 4b, FIGS. 4a and 4b shows a principle of the model combination technology of the graph computation technology. In FIG. 4a, a common vertex is searched between the two sub-graphs (the sub-graph of doctor and patient and the sub-graph of doctor and medical advice). For example, two same doctor vertexes in a dashed box of the FIG. 4a are searched, which are a doctor vertex 2 and a doctor vertex 3, other vertexes related to the searched doctor vertexes are retained, which are a patient vertex 1, a patient vertex 3, a patient vertex 4, a medical advice 1 and a medical advice 2. The sub-graphs are fused to obtain the fused large graph. The fused large graph is shown as FIG. 4b. Therefore, the sub-graph of doctor and patient and the sub-graph of doctor and medical advice are fused to form the fused large graph according to the model combination technology.

It needs to be explained that, FIGS. 2, 3, and 4b are not used for limited the formation of the sub-graph of doctor and patient, the sub-graph of doctor and medical advice, and the fused large graph. The relationship between the amount and the vertexes of the sub-graph of doctor and patient, the sub-graph of doctor and medical advice, and the fused large graph can be different according to an actual situation.

The foregoing disclosure establishes a medical graph data using the graph computation technology. The structured data stored in the traditional database are stripped to form entities and relationships. The entities and the relationships are mapped into vertexes and edges. The vertexes and the edges are converted into graph data to be stored in the network. Thus, a claim settlement anti-fraud method according to the medical graph data is achieved.

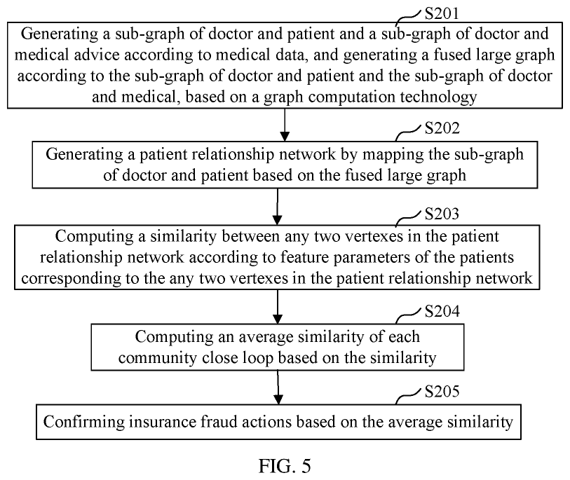

Referring to FIG. 5, FIG. 5 shows a flowchart of the claim settlement anti-fraud method of the present disclosure based on the graph computation technology. The claim settlement anti-fraud method is aimed to the patients. By using the claim settlement anti-fraud method, patient frauds are identified, especially patient group fraud actions. Thus, illegal and criminal behaviors are combated, and security financial are better maintained.

As shown in FIG. 5, the claim settlement anti-fraud method based on the graph computation technology includes steps S201 to S205.

In block S201, generating the sub-graph of doctor and patient and the sub-graph of doctor and medical advice according to the medical data, and generating a fused large graph according to the sub-graph of doctor and patient and the sub-graph of doctor and medical advice, based on the graph computation technology.

In detail, the sub-graph of doctor and patient are generated according to the patient and doctor relationship table, the sub-graph of doctor and medical advice are generated according to the doctor and medical relationship table, based on the graph computation technology. The fused large graph is generated according to the sub-graph of doctor and patient and the sub-graph of doctor and medical advice based on the model combination technology of the graph computation technology.

In block S202, generating a patient relationship network by mapping the sub-graph of doctor and patient according to the fused large graph.

The patient relationship network includes a plurality of community close loops. Different community close loops represent different communities. There are a plurality of patients and doctors in different communities.

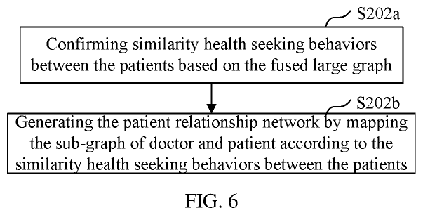

In one embodiment, as shown in FIG. 6, the step of generating the patient relationship network by mapping the sub-graph of doctor and patient includes sub-steps from S202a to a sub-step S202b.

In block S202a, confirming similarity health seeking behaviors between the patients according to the fused large graph.

The similarity health seeking behaviors mean that a same doctor is visited by different patients in patient's community of the doctor. When several patients visit the same doctor, which is considered as a similarity health seeking behavior.

For example, as shown in FIG. 4b, the similarity health seeking behaviors are confirmed by the fused large graph. In detail, both the patient 1 and the patient 3 visit the same doctor 2, the patient 1 and the patient 4 visit the same doctor 3, therefore, a similarity health seeking behavior is confirmed between the patient 1 and the patient 4, and a similarity health seeking behavior is confirmed between the patient 1 and the patient 4. There is no same doctor visited by the patient 3 and the patient 4, thus there is no similarity health seeking behavior between the patient 3 and the patient 4.

In block S202b, generating the patient relationship network by mapping the sub-graph of doctor and patient according the similarity health seeking behaviors between the patients.

In detail, a visiting time of two patients visiting a common doctor is conformed according to the similarity health seeking behavior. For example, in the sub-graph of doctor and patient as shown in FIG. 2, both the patient 2 and the patient 4 visit the doctor 1 and the doctor 2, the visiting time of the patient 2 and the patient 4 are 2. Both the patient 3 and the patient 7 visit the doctor 4, the visiting time of the patient 3 and the patient 7 is 1.

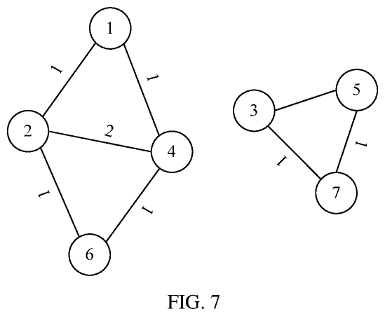

Therefore, the patient relationship network is generated by mapping the sub-graph of doctor and patient according the graph computation technology. The mapped patient relationship network as shown in FIG. 7. In FIG. 7, there are 7 vertexes, which is the patient 1, the patient 2, the patient 3, the patient 4, the patient 5, the patient 6, and the patient 7. Each connected line between the patients represents the similarity health seeking behaviors between the patients. The number on each connection line represents a weight. The weight represents the visiting time of the patients visiting a common doctor. For example, the visiting time of the patient 2 and the patient 4 visiting the common doctor is 2.

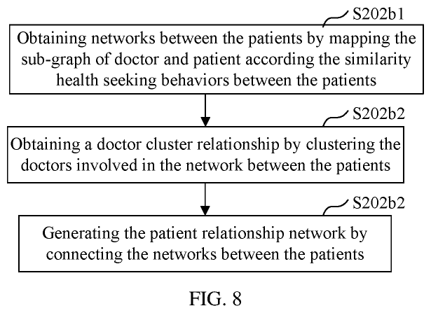

In one embodiment, due to increase an accuracy of the claim settlement anti-fraud method, the doctors need to be clustered for improving the patient relationship network. Based on this, as shown in FIG. 8, the step of S202b includes sub-steps S202b1 to S202b3.

In block S202b1, obtaining networks between the patients by mapping the sub-graph of doctor and patient according the similarity health seeking behaviors between the patients. In block S202b2, obtaining a doctor cluster relationship by clustering the doctors involved in the networks between the patients. In block S202b3, generating the patient relationship network by connecting the networks between the patients.

The networks between the patients are obtained by mapping the sub-graph of doctor and patient according to the similarity health seeking behaviors between the patients. For example, as shown in FIG. 7, there are two networks between the patients, a networks between the patients is composed by the patient 1, the patient 2, the patient 4, and the patient 6, and a network between the patients is composed by the patient 3, the patient 5, and the patient 7.

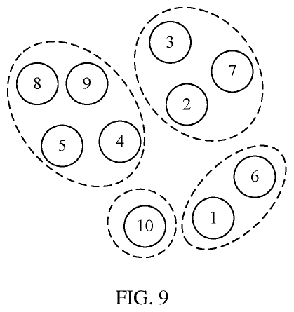

The departments of the doctors need to be considered while clustering the involved doctors in the networks between the patients for obtaining a doctor clustering relationship. Because, the doctor and the department are in a one-to-many relationship. The doctors are clustered according the departments, and the result of clustering the doctor is shown as FIG. 9 according to the departments. For example, the doctor 4, the doctor 5, the doctor 8, and the doctor 9 belong to a common department, the doctor 2, the doctor 3, and the doctor 7 belong to another department. The clustering relationship is the belong department.

It needs to be explained that, the doctor cluster in FIG. 9 includes 10 doctors, it is not clustered the doctor in FIG. 7. FIG. 9 is merely an example for explaining the cluster of the doctor. The clustering of the doctor in FIG. 7 can references the above clustering manner.

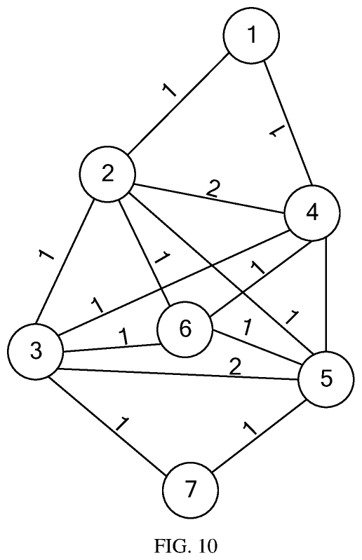

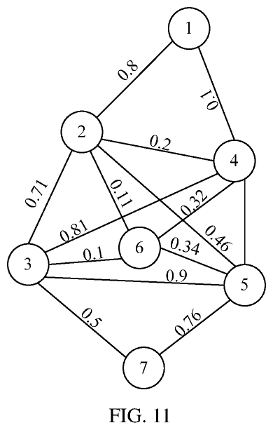

The final patient relationship network is generated by connecting the networks between the patients according to the doctor clustering relationship. The final patient relationship network is shown in FIG. 10 or FIG. 11. The final patient relationship network includes a plurality of community close loops. The community close loops are strongly connected structure (a close loop composed by several vertexes, and there is an edge between any two vertexes in the close loop, as shown in FIG. 10, there are three close loops structure, which are a close loop of 1-2-4, a close loop of 2-3-4-5-6, and a close loop of 3-5-7). Different close loop structures represent different communities. Residents in the community visits a common doctor (or a common category doctor), and have a similarity health seeking behavior.

In block S203, a similarity between any two vertexes in the patient relationship network is computed according to feature parameters of patients corresponding to the any two vertexes in the patient relationship network.

The feature parameters of the patients corresponding to the any two vertexes in the patient relationship network include feature attributes and health seeking behavior attributes. The feature attributes include an age of the patient. The health seeking behavior attributes include a number of the medical advices and a medical cost of the patient.

In a selectable embodiment, the feature attributes and the health seeking behavior attributes can also include other parameters, such as a sex of the patient, a category of the medical advice, and so on.

In detail, according to a similarity computation formula, the similarity of the any two vertexes in the patient relationship network are computed according the corresponding feature attributes and the health seeking behavior attributes of the corresponding patients.

The similarity computation formula is shown as below.

$$sim 〈 A , B 〉 = \frac{{{\sum }^{n = 3}}_{i = 1} {A}_{i} {B}_{i}}{\sqrt{{{\sum }^{n = 3}}_{i = 1} {A}_{i}^{2}} \sqrt{{{\sum }^{n = 3}}_{i = 1} {B}_{i}^{2}}} \qquad ( 1 )$$

In the above similarity computation formula, sim <A, B> represents the similarity, which is a cosine similarity, A and B represent the corresponding patients. A₁ represents an age of the patient A, A₂ represents a number of the medical advices of the patient A. A₃ represents a medical cost of the patient A. B₁ represents an age of the patient B. B₂ represents a number of the medical advices of the patient B. B₃ represents a medical cost of the patient B.

Thus, according to the similarity computation formula, the similarity of the any two vertexes in the patient relationship network are computed according the corresponding feature attributes and the health seeking behavior attributes of the corresponding patients.

In block S204, an average similarity of each community close loop is computed according to the similarity.

In detail, according to an average similarity computation formula, the average similarity of each community close loop is computed according to the similarity.

The average similarity computation formal is shown as below.

$$ϕ ( P ) = \frac{{{\sum }^{n = N}}_{i = 1} {w}_{t}}{N} \qquad ( 2 )$$

In the above average similarity computation formula, ϕ(P) represents the average similarity of the close loop of the community. P represents the community. N represents path coefficient of the community, and is a positive integer. W represents a weight of the path coefficient.

In detail, the similarity computation formula is used for computing the similarity between any two vertexes of the patient relationship network. According to the similarity computation formula computes the similarity between any two patients in the community as shown in FIG. 10, and the weight between the corresponding two patients in the FIG. 10 is updated according to the computed similarity. The patient relationship network updating by the acquired weight is shown in FIG. 11.

Correspondingly, the step of S204 includes computes the average similarity of the close loops in each community according the updated weight of the patient relationship network according to the average similarity computation formula. Of course, the average similarity computation formula is used for computing the average similarity. In the patient relationship network with the updated weight, the average similarities of different community can be quantitatively computed, which means a uniformity behavior.

For example, the community 3-5-7, according to the average similarity computation formula, the average similarity of the community is

$$ϕ ( P ) = \frac{0.9 + 0.5 + 0.76}{3} = 0.72 ,$$

the average similarity of the community 3-5-7 is 0.72. The average similarities of other communities also can be computed, and the average similarities are ranked putted in numerical order. As shown in FIG. 6, the average similarities of different communities ranking table is shown as below.

TABLE 6
Average similarities of different communities
ranking table

| Community name | Average similarity ϕ |
|---|---|
| 3-5-7 | 0.72 |
| 2-3-4-5-6 | 0.41 |
| 1-2-4 | 0.37 |

In block 205, insurance fraud actions are confirmed according to the average similarity.

In detail, the communities are sorted according to the average similarities, and insurance fraud groups with a high suspicion are confirmed according to the numerical sequence to provide an important reference for screening frauds. The patient groups usually have typical characteristics of high consistent behaviors. Based on miming suspicion group by community clustering, different communities are divided according to the health seeking behaviors of the patients. The average similarity of each community is computed by the similarities of the health seeking behaviors of the patients in same community. Therefore, the consistency of collective behavior of the community can be measured according the average similarity to quickly confirm insurance fraud.

The claim settlement anti-fraud method based on the graph computation technology of the above embodiment generates the sub-graph of doctor and patient, the sub-graph of doctor and medical advice, and the fused large graph according to the medical data. A patient relationship network is generated by mapping the sub-graph of doctor and patient according to the fused graph. The patient relationship network includes a plurality of community close loops. The similarity between any two vertexes in the patient relationship network are computed according to feature parameters of patients corresponding to the any two vertexes in the patient relationship network. The average similarity of each community close loop is computed according to the similarity. The insurance fraud actions are confirmed according to the average similarity. Therefore, insurance fraud patient groups with high suspicion are confirmed to provide the important reference for quickly confirming the insurance fraud.

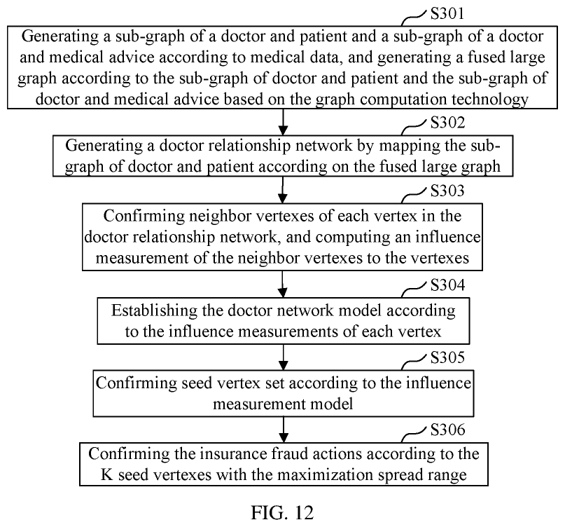

Referring to FIG. 12, FIG. 12 illustrates another embodiment of the claim settlement anti-fraud method based on the graph computation technology. The claim settlement anti-fraud method is aimed to doctors. Doctor fraud are confirmed by the claim settlement anti-fraud method, thus illegal and criminal behaviors are combated, and security financial are better maintained.

As shown in FIG. 12, the claim settlement anti-fraud method based on the graph computation technology specifically includes steps from S301 to S306.

In block S301, generating a sub-graph of doctor and patient and a sub-graph of doctor and medical advice according to medical data, and generating a fused large graph according to the sub-graph of doctor and patient and the sub-graph of doctor and medical advice based on the graph computation technology.

In detail, based on the graph computation technology, the sub-graph of doctor and patient are generated according to the patient and doctor relationship table, the sub-graph of doctor and medical advice are generated according to the doctor and medical advice relationship table. The fused large graph is fused according to the sub-graph of doctor and patient and the sub-graph of doctor and medical advice.

In block S302, generating a doctor relationship network by mapping the sub-graph of doctor and patient according on the fused large graph.

Besides the insurance fraud of the patients, doctors also can use their influence to defraud. Main manifestations include a large number of patients visiting, a large number of medical advices, a large amount of medication, and so on.

Due to the number of the patient visiting, the more patients a doctor accepts in a time duration, the more influential of the doctor is in a professional field. Due to the number of the medical advices and the amount of the medication, the number of the medical advices and the amount of the medication can quantify a doctor's workload, and reflects the influence of the doctor. Thus, the doctor relationship can be used to computed the influence of the doctor to confirm insurance fraud.

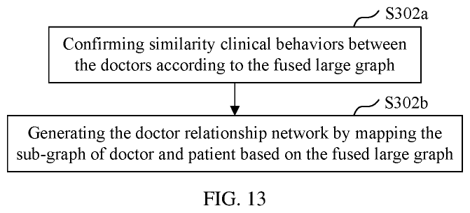

In detail, as shown in FIG. 13, the step of generating a doctor relationship network by mapping the sub-graph of doctor and patient according on the fused large graph can include sub-steps of S302a and S302b.

In block S302a, confirming similarity clinical behaviors between the doctors according to the fused large graph; in block S302b, generating the doctor relationship network by mapping the sub-graph of doctor and patient based on the fused large graph.

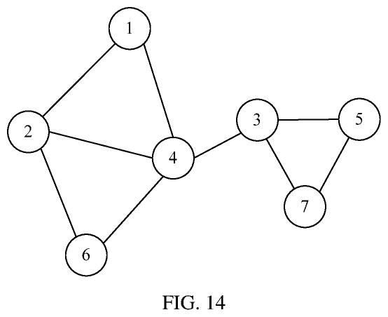

A similarity clinical behavior means that two doctors accept a common patient. It also can be other behaviors, such as seeing patients in a common family or in a common community. The doctor relationship network is generated by mapping the sub-graph of doctor and patient by the graph computation technology based on the similarity clinical behaviors. The generated doctor relationship network is shown as FIG. 14. For example, the doctor 4 and the doctor 3 accept a same patient.

A doctor network model is generated according to the doctor relationship network. The doctor network model is represented as G=<V,E>. V represents a doctor vertex set, and E represents an edge formed by the two doctors accepting a common patient. In detail, the doctor relationship network is shown in FIG. 14.

In block S303, confirming neighbor vertexes of each vertex in the doctor relationship network, and computing an influence measurement of the neighbor vertexes to the vertexes.

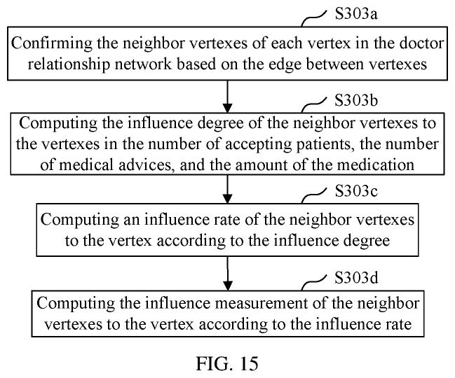

In detail, as shown in FIG. 15, the step of confirming neighbor vertexes of each vertex in the doctor relationship network can include sub-steps from S303a to S303d.

In block S303a, confirming the neighbor vertexes of each vertex in the doctor relationship network based on the edge between vertexes.

For example, as shown in FIG. 14, there is edges between the vertex 4 and the vertex 1, the vertex 2, the vertex 3, and the vertex 6. Thus, there are four neighbor vertexes corresponding to the vertex 4. In a same way, the vertex 1 includes two neighbor vertexes, the vertex 2 includes three neighbor vertexes, the vertex 3 includes three neighbor vertexes, the vertex 5 includes two neighbor vertexes, the vertex 6 includes two neighbor vertexes, and the vertex 7 includes two neighbor vertexes.

In block S303b, computing the influence degree of the neighbor vertexes to the vertexes in the number of accepting patients, the number of medical advices, and the amount of the medication.

In detail, by influence degree computation formulas, the influence degree of the neighbor vertexes to the vertexes are computed. The influence degree computation formulas are shown as below.

$$\begin{matrix}A c c ( i , j ) = \frac{\vert {T}_{j} \vert}{{\sum }_{a ∈ A ( i )} \vert {T}_{a} \vert} & ( 3 ) \\ Am o ( i , j ) = \frac{\vert {Z}_{j} \vert}{{\sum }_{{ }_{a ∈ A ( i )}} \vert {Z}_{a} \vert} & ( 4 ) \\ Fin ( i , j ) = \frac{\vert {M}_{j} \vert}{{\sum }_{a ∈ A ( i )} \vert {M}_{a} \vert} & ( 5 )\end{matrix}$$

Acc(i, j) represents an influence degree of the vertex j to the vertex i in the number of accepting patients. The vertex j is the neighbor vertex to the vertex i. Amo(i, j) represents an influence degree of the vertex j to the vertex i in the number of medical advices. Fin(i, j) represents an influence degree of the vertex j to the vertex i in the amount of medication. |Tⱼ| represents a total number of accepting patients to the vertex j. Σ_{a∈A(i)}|Tₐ| represents a total number of accepting patients of the neighbor vertex to the vertex i. |Zⱼ| represents a total number of the medical advices of the vertex j. Σ_{a∈A(i)}|Zₐ| represents a total number of the medical advices of the neighbor vertexes to the vertex i. |Mⱼ| represents a total amount of the medication of the doctor j. Σ_{a∈A(i)}|Mₐ| represents a total amount of the medication of the neighbor vertexes to the vertex i.

In block S303c, computing an influence rate of the neighbor vertexes to the vertex according to the influence degree.

The neighbor vertexes to the vertex I are defined by A(i)={j|(i, j)}. For measuring an influence capacity of the neighbor vertexes to the vertex, the influence rate represents the influence capacity.

In detail, the step of computing the influence rate of the neighbor vertexes to the vertex according to the influence degree includes the influence rate of the neighbor vertexes to the vertex are computed according to an influence rate computation formula.

The influence rate computation formula is shown as below.

I(i,j)=Acc(i,j)*Amo(i,j)*Fin(i,j) (6)

In the influence rate computation formula, I(i, j) represent the influence rate, Acc(i, j) represents an influence degree of the vertex j to the vertex i in a number of accepting patients. Amo(i, j) represents an influence degree of the vertex j to the vertex i in a number of the medical advices. Fin(i, j) represents an influence degree of the vertex j to the vertex i in an amount of the medication.

In block S303d, computing the influence measurement of the neighbor vertexes to the vertex according to the influence rate.

In detail, the step of the computing the influence measurement of the neighbor vertexes to the vertex according to the influence rate includes the influence measurement of the neighbor vertexes to the vertex being iteration computed according to the influence rate based on an influence measurement computation formula.

The influence measurement computation formula is shown as below.

$$\begin{matrix}DIR ( i ) = d + ( 1 - d ) {\sum }_{j ∈ N ( i )} [ {S}_{i j} * DI R ( j ) ] & ( 6 ) \\ {S}_{i j} = \frac{I ( i , j )}{{\sum }_{a ∈ A ( i )} I ( i , a )} & ( 7 )\end{matrix}$$

DIR(i) represents an influence measurement of the vertex i. N(i) represents neighbor vertex set of the vertex i. Sᵢⱼ represents a scale factor of the vertex j being allocated by the influence of the vertex i. I(i, j) represents a proportion of the vertex j in all the neighbor vertexes to the vertex i. d represents a damping factor, and is a constant. Σ_{a∈A(i)}I(i, a) represents a sum of the influence rate of the neighbor vertexes of the vertex i. a represents all the neighbor vertexes of the vertex i, and a positive integer.

In one embodiment, the damping factor d is set at 0.85, an initial value of DIR is 0.1. All DIR values of the whole doctors are obtained by iteration computing.

In block 304, establishing the doctor network model according to the influence measurements of each vertex.

In detail, the step of the doctor network model according to the influence measurements of each vertex includes computing the influence weight of each edge in the doctor relationship network according to the influence measurement of each vertex, and establishing a linear threshold model according to the computed influence weight. The linear threshold model is used for confirming activation vertex.

A maximization problem of an influence defines as how to select K initial vertexes to maximize a final spread influence range. By computing the influence measurement (the value of the DIR) of the doctor, an influence ranking of the doctors is obtained. If the top K vertexes are directly selected as the initial vertexes, the maximizing of the final spread influence range cannot be ensured. Because some departments are more popular, it will cause that the K vertexes are gathered in a common cluster, and other weak connected vertexes in the doctor relationship network are ignored. Thus, the DIR values ranking easily make the doctors in the popular departments to top, but the spread influence range cannot be maximized.

In one embodiment, in order to accurately confirm the K vertexes for maximizing the influence spread range, an influence spread model is established, and is a linear threshold model. In other embodiments, the influence spread model also can be other type models, such as an independent cascade model.

In the doctor relationship network, the doctor with a higher influence can influence to neighbor doctor, and the spread of the influence depends on whether the doctor of the neighbor vertex is active. The linear threshold model is established for predicting a situation of the influence by the computed influence weight.

In detail, a given doctor network model G=<V,E>. N(v) is defined as a neighbor vertex set to the vertex v. An influence of the activated vertex u to the neighbor vertex u is bᵤᵥ. The bᵤᵥ is the influence weight. A sum of the influence of the vertex v to all the neighbor vertexes is less than 1. A(v) is defined as an activated neighbor vertex set in the neighbor vertexes to the vertex v. A threshold θᵥ is preset to each vertex. θᵥ represents an empirical value, and is set according to an actual experience. When the influence weight bᵤᵥ is larger than the threshold θᵥ, the vertex V is active.

The bᵤᵥ in the linear threshold model represents the influence of the activated vertex u to the neighbor vertex v, which is the influence weight. The influence weight is computed by the following computation formula.

$${b}_{u v} = \frac{D I R ( u )}{{\sum }_{a ∈ N ( v )} DIR ( a )} \qquad ( 8 )$$

DIR(u) represents the influence measurement of the vertex u. N(v) represents a neighbor vertex set of the vertex v. bᵤᵥ represents the influence weight, and reflects the proportion of the influence of the vertex u in the set N(v). A probability of vertex v being activated depends on an influence of the activated vertexes in the set N(v). The greater the influence, the greater probability of the vertex v being activated.

In block S305, confirming seed vertex set according to the influence measurement model.

The seed vertex set includes the K seed vertexes with the maximum spread ranges. K is a positive integer. The K seed vertexes means the influence spread range of the K seed vertexes being maximized, which are wider than the spread ranges of other vertexes.

In detail, the step of confirming the seed vertex set according the influence model includes circularly computing the vertexes with the maximization increased of the influence spread range in each selecting step by the influence measurement model based on a greedy algorithm, for obtaining the seed vertex set is obtained.

The influence maximization algorithm of the doctor relationship network can be achieved by the greedy algorithm, and confirm the K seed vertexes with the widest spread orientation. Core steps of the algorithm is: circularly computing the vertexes with the maximization increased of the influence spread range in each selecting step by the greedy algorithm according the established linear threshold model, and finally obtaining the seed vertex set.

For example, the doctor network G=<V,E> is defined. S represents a seed set comprising the K vertexes. Sv represents spread range obtained by once spreading. IS{S} represents a final influence range of the seed set S.

Using the greedy algorithm, pseudocodes of the seed vertex set being final dug are shown as below:

```
Input : doctor network G=<V,E>
Output: K seed vertexes
  Initialization S= [symbol] , R=1000
  for v in V:
    return DIR(v)
  for edge(u,v) in E:
    return buv
  for i in range(K):
    for v in V:
      sv=0
      for j in range(R):
        sv += |IS{v}|
    sv=sv/R
  return S=S{argvmax{sv} }
```

By circularly computing the vertexes with the maximization increased of the influence spread range, the final seed vertex set is obtained. The seed vertex set includes the K seed vertexes with maximization spread range.

In block S306, confirming the insurance fraud actions according to the K seed vertexes with the maximization spread range.

In one case of a medical insurance fraud, there are some doctors involved in the insurance fraud. By establishing a model for the doctor relationship network from a doctor influence spread angle, the K seed vertexes with higher influence and the maximization spread range in the doctor relationship network are obtained. The actions of the doctors corresponding to the K seed vertexes can be the insurance fraud actions. Thus, the doctors are insurance fraud doctors. Therefore, it provides an important reference for identifying doctor fraud.

The claim settlement anti-fraud method based on the graph computation technology of the above embodiments generates the sub-graph of doctor and patient, the sub-graph of doctor and medical advice, and the fused large graph according to medical data. The doctor relationship network is generated by mapping the sub-graph of doctor and patient according to the fused large graph. The influence measurement model is established based on the doctor relationship network. The insurance fraud actions are confirmed by the influence measurement model. The insurance fraud doctors with the high suspicion are obtained to provide the important refence for quickly identifying insurance fraud actions.

It needs to explain that, the claim settlement anti-fraud method of FIG. 5 and FIG. 12 can be separately used for identifying the insurance fraud action of the patients or the doctors, and also can be cooperated used for identifying the insurance fraud action of the patients and the doctors. The illegal and criminal behaviors are combated, and security financial are better maintained.

For example, the present disclosure also provides a third embodiment of the claim settlement anti-fraud method. The method includes the following steps:

The sub-graph of doctor and patient and the sub-graph of doctor and medical advice according to medical data based on a graph computation technology, and the fused large graph are generated according to the sub-graph of doctor and patient and the sub-graph of doctor and medical advice. The patient relationship network and the doctor relationship network are generated by mapping the sub-graph of doctor and patient according the fused large graph. The patient relationship network includes the plurality of community close loops. The similarity between any two vertexes of the patient relationship network is computed according to the feature parameters of the patients corresponding to the any two vertexes of the patient relationship network. The average similarity of each community close loop is computed according to the similarity. The neighbor vertexes of each vertex in the doctor relationship network are confirmed. The influence measurement of the neighbor vertexes to the vertex is computed. The influence measurement model is established according to the influence measurement of each vertex. The seed vertex set is confirmed according to the influence measurement model. The seed vertex set includes K seed vertexes with the maximization spread range. K is a positive integer. The insurance fraud actions are confirmed by the average similarity and/or the K seed vertexes with the maximization spread range.

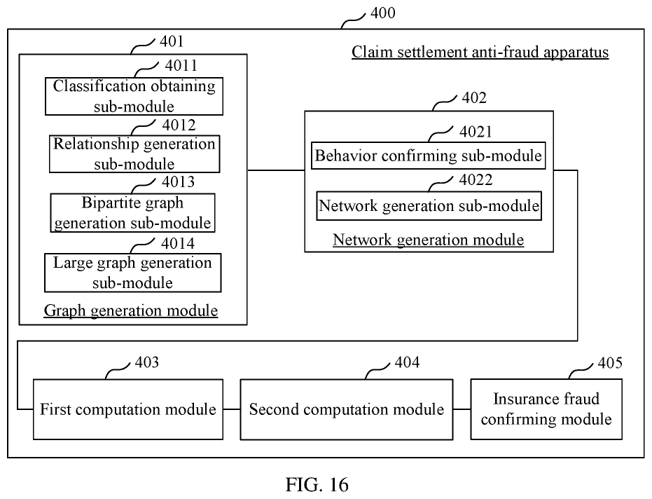

Referring to FIG. 16, FIG. 16 illustrates a claim settlement anti-fraud apparatus. The claim settlement anti-fraud apparatus executes the foregoing claim settlement anti-fraud methods. The claim settlement anti-fraud apparatus can be deployed in a server or a terminal.

The server can be a dependent server, and also can be a server group. The terminal can be mobile, a tablet personal computer, a notebook, a desk computer, a personal assistance digital, a wearable device, and so on.

As shown in FIG. 16, the claim settlement anti-fraud apparatus 400 includes a graph generation module 401, a network generation module 402, a first computation module 403, a second computation module 404, and an insurance fraud confirming module 405.

The graph generation module 401, configured to generate a sub-graph of doctor and patient and a sub-graph of doctor and medical advice according to medical data, and generate a fused large graph according to the sub-graph of doctor and patient and the sub-graph of doctor and medical advice, based on a graph computation technology.

The graph generation module 401 includes a classification obtaining sub-module 4011, a relationship generation sub-module 4012, a bipartite graph generation sub-module 4013, and a large graph generation sub-module 4014.

In detail, the classification obtaining sub-module 4011 is configured to acquire the medical data and classify into classified data. The relationship generation sub-module 4012 is configured to generate a classification relationship table according to association relationships of the classification data. The bipartite graph generation sub-module 4013 is configured to generate a bipartite graph according to the classification relationship table by a graph computation technology. The large graph generation sub-module 4014 is configured to generate a fused large graph according to the graph of the doctor and patient and the sub-graph of doctor and medical advice by a model combination technology of the graph computation technology.

The network generation module 402 is configured to generate a patient relationship network by mapping the sub-graph of doctor and patient according to the fused large graph. The patient relationship network includes a plurality of community close loops.

The network generation module 402 further includes a behavior confirming sub-module 4021 and a network generation sub-module 4022. The behavior confirming sub-module 4021 is configured to confirm similarity health seeking behaviors between the patients according to the fused large graph. The network generation sub-module 4022 is configured to generate the patient relationship network by mapping the sub-graph of doctor and patient according the similarity health seeking behaviors between the patients.

In one embodiment, the network generation sub-module 4022 is configured to obtain networks between the patients by mapping the sub-graph of doctor and patient according the similarity health seeking behaviors between the patients, obtain a doctor cluster relationship by clustering the doctors involved in the networks between the patients, and generate the patient relationship network by connecting the networks between the patients.

The first computation module 403 is configured to compute a similarity between any two vertexes in the patient relationship network according to feature parameters of the corresponding two vertexes of the corresponding patients.

In detail, the first computation module 403 is configured to compute the similarity between any two vertexes in the patient relationship network according to corresponding feature attributes and the health seeking behavior attributes of the corresponding patients, based on a similarity computation formula.

In one embodiment, the first computation module 403 also is configured to update weight of each edge in the patient relationship network according to the similarity.

The second computation module 404 is configured to compute an average similarity of each community close loop according to the similarity.

In detail, the second computation module 404 is configured to compute the average similarity of each community close loop according to the weight in the patient relationship network based on an average similarity computation formula.

Accordingly, the second computation module 404 is configured to compute the average similarity of each community close loop according to the updated weight in the patient relationship network, based on the average similarity computation formula.

The insurance fraud confirming module 405 is configured to confirm the insurance fraud actions according to the average similarity.

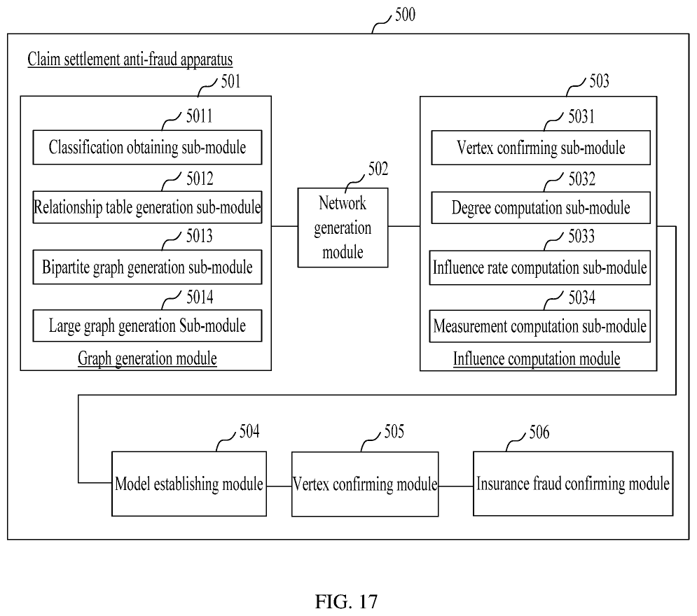

Referring to FIG. 17, FIG. 17 illustrates another embodiment of the claim settlement anti-fraud apparatus based on a graph computation technology. The claim settlement anti-fraud apparatus is configured to execute the foregoing claim settlement anti-fraud method based on the graph computation technology. The claim settlement anti-fraud apparatus is deployed in a server.

As shown in FIG. 17, the claim settlement anti-fraud apparatus 500 includes a graph generation module 501, a network generation module 502, a fluence computation module 503, a model establishing module 504, a vertex confirming module 505, and an insurance fraud confirming module 505.

The graph generation module 501, configured to generate a sub-graph of doctor and patient and a sub-graph of doctor and medical advice according to medical data, and generate a fused large graph according to the sub-graph of doctor and patient and the sub-graph of doctor and medical advice, based on a graph computation technology.

In one embodiment, the graph generation module 501 includes a classification obtaining sub-module 5011, a relationship table generation sub-module 5012, a bipartite graph generation sub-module 5013, and a large graph generation sub-module 5014.

In detail, the classification obtaining sub-module 5011 is configured to acquire the medical data and classify into classified data. The relationship table generation sub-module 5012 is configured to generate a classification relationship table according to association relationships of the classification data. The bipartite graph generation sub-module 5013 is configured to generate a bipartite graph according to the classification relationship table by a graph computation technology. The large graph generation sub-module 5014 is configured to generate a fused large graph according to the graph of the doctor and patient and the sub-graph of doctor and medical advice by a model combination technology of the graph computation technology.

The network generation module 502 is configured to generate a doctor relationship network by mapping the sub-graph of doctor and patient according to the fused large graph.

In detail, in one embodiment, the network generation module 502 is configured to confirm similarity health seeking behaviors based on the fused large graph, and generate the doctor relationship network by mapping the sub-graph of doctor and patient according to the similarity health seeking behaviors.

The influence computation module 503 is configured to confirm neighbor vertexes of each vertex in the doctor relationship network, and compute influence measurement of the neighbor vertexes to the vertex.

In one embodiment, the influence computation module 503 includes a vertex confirming sub-module 5031, a degree computation sub-module 5032, an influence rate computation sub-module 5033, and a measurement computation sub-module 5034.

In detail, the vertex confirming sub-module 5031 is configured to confirm the neighbor vertexes of each vertex in the doctor relationship network based on the edge between vertexes. The degree computation module 5032 is configured to compute the influence degree of the neighbor vertexes to the vertexes in the number of accepting patients, the number of medical advices, and the amount of the medication. The influence rate computation sub-module 5033 is configured to compute an influence rate of the neighbor vertexes to the vertex according to the influence degree. The measurement computation sub-module 5034 is configured to compute the influence measurement of the neighbor vertexes to the vertex according to the influence rate.

The model establishing module 504 is configured to establish the doctor network model according to the influence measurements of each vertex.

In detail, in one embodiment, the model establishing module 504 is configured to compute the influence weight of each edge in the doctor relationship network according to the influence measurement of each vertex, and establish a linear threshold model according to the computed influence weight.

The vertex confirming module 505 is configured to confirm seed vertex set according to the influence measurement model.

In detail, the vertex confirming module 505 is configured to circularly compute the vertexes with the maximization increased of the influence spread range in each selecting step by the influence measurement model based on a greedy algorithm, for obtaining the seed vertex set is obtained.

The insurance fraud confirming module 506 is configured to confirm the insurance fraud actions according to the K seed vertexes with the maximization spread.

It needs to explained that, for the convenience and simplicity of the description, it is clearly and understandable to those of an ordinary skill in the art that the specific working process of the foregoing claim settlement anti-fraud apparatus based on the graph computation technology and all the modules can references the claim settlement anti-fraud method based on the graph computation technology. There is no need to repeated here.

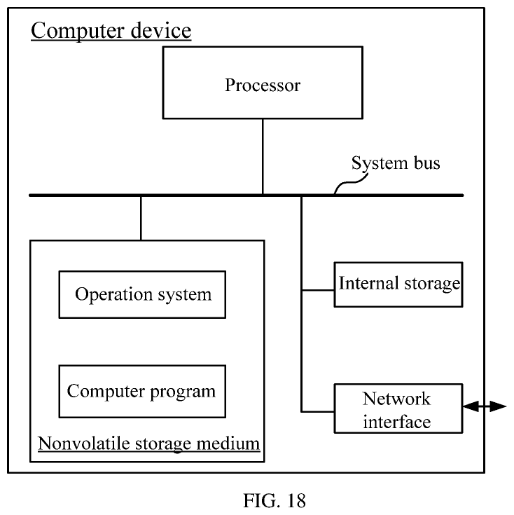

The foregoing claim settlement anti-fraud apparatus can execute in a manner of computer programs. The computer programs can be implemented in the computer device as shown in FIG. 18.

Referring to FIG. 18, FIG. 18 is a structural diagram view of a computer device of the present disclosure. The computer device can be a server or a terminal.

Referring to FIG. 18, the computer device includes a processor, a storage, and a network interface, which are connected through a system bus. The storage can include a non-volatile storage and an internal storage.

The non-volatile storage can store an operation system and computer programs. The computer programs incudes program instructions. When the computer instructions being executed, the processor implements a claim settlement anti-fraud method.

The processor is configured to provide computation and control ability, for supporting an operation of the computer device.

The internal storage provides an operation environment of the computer instructions of the non-volatile storage. When the computer instructions being executed, the processor implements a claim settlement anti-fraud method.

The network interface is configured to provide a network communication, such as sending an assigned task, and so on. It is understood that, the structure as shown in FIG. 18 is a partial structure related to the present disclosure, and are not intended to limit the computer device applied the present disclosure. The computer device can include more or less components, or some components being combined, or different components.

It is understood that, the processor can be a central processing unit (CPU), and can be other general processor, a digital signal processor (DSP), an application specific integrated circuit (ASIC), a field programmable gate array (FPGA), or other programmable logic device, a separate gate or transistor logic device, a separate hardware component, and so on. The general processor can be a microprocessor, other regular data processing chips, and so on.

The present disclosure also provides a computer readable storage medium. The computer readable storage medium stores computer programs. The computer programs incudes program instructions. The processor executes the program instructions to implement any claim settlement anti-fraud method based on the graph computation technology of the present disclosure.

The computer readable storage medium can be an internal storage of the foregoing computer device, such as a hard disk or a memory. The computer readable storage medium also can be external storage device of the computer device, such as a plug-in hard disk in the computer device, a smart media card (SMC), a secure digital (SD), a flash card, and so on.

The foregoing implementations are merely preferably embodiments of the present disclosure, and are not intended to limit the protection scope of the present disclosure. Any equivalent structure variation using the present disclosure and drawings, being directly or indirectly used in other related technical fields shall all fall into the protection scope of the present disclosure. Thus, the protection scope of the present disclosure shall be subjected to the protection scope of the claims.

What is claimed is:

1. A claim settlement anti-fraud method, wherein the method comprising:
generating a sub-graph of doctor and patient a sub-graph of doctor and medical advice according to medical data, and generating a fused large graph according to the sub-graph of doctor and patient and the sub-graph of doctor and medical advice, based on the graph computation technology;
generating a patient relationship network by mapping the sub-graph of doctor and patient according to the fused large graph; the patient relationship comprises a plurality of community close loops;
using a similarity computation formula, computing a similarity between any two vertexes in the patient relationship network according to feature parameters of the patients corresponding to the any two vertexes in the patient relationship network;
using an average similarity computation formula, computing an average similarity of each community close loop according to the similarity; and
confirming insurance fraud actions according to the average similarity;
wherein the similarity computation formula is:
$$sim 〈 A , B 〉 = \frac{{{\sum }^{n = 3}}_{i = 1} {A}_{i} {B}_{i}}{\sqrt{{{\sum }^{n = 3}}_{i = 1} {A}_{i}^{2}} \sqrt{{{\sum }^{n = 3}}_{i = 1} {B}_{i}^{2}}}$$
Wherein sim <A, B> represents the similarity, which is a cosine similarity, A and B represent the corresponding patients; A1 represents an age of the patient A, A2 represents a number of the medical advices of the patient A; A3 represents a medical cost of the patient A; B1 represents an age of the patient B; B2 represents a number of the medical advices of the patient B; A3 represents a medical cost of the patient B;
wherein the average similarity computation formula is:
$$ϕ ( P ) = \frac{{{\sum }^{n = N}}_{i = 1} {w}_{i}}{N}$$
wherein ϕ(P) represents the average similarity of the close loop of the community; P represents the community; N represents path coefficient of the community, and is a positive integer; W represents a weight of the path coefficient.

2. The claim settlement anti-fraud method of claim 1, wherein the step of generating a sub-graph of doctor and patient a sub-graph of doctor and medical advice according to medical data, and generating a fused large graph according to the sub-graph of doctor and patient and the sub-graph of doctor and medical advice, based on the graph computation technology further comprising:
classifying the acquired medical data to obtain classified data;
generating a classification relationship table according to association relationships of the classification data; the classification relationship table comprises a patient and doctor relationship table and a doctor and medical advice relationship table;
generating a bipartite graph according to the classification relationship table by a graph computation technology; and
generating the fused large graph according to the sub-graph of doctor and patient and the sub-graph of doctor and medical advice by a model combination technology of the graph computation technology.

3. The claim settlement anti-fraud method of claim 1, wherein the step of generating a patient relationship network by mapping the sub-graph of doctor and patient according to the fused large graph further comprising:
confirming similarity health seeking behaviors between the patients; and
generating the patient relationship network by mapping the sub-graph of doctor and patient according to the similarity health seeking behaviors between the patients.

4. The claim settlement anti-fraud method of claim 3, wherein the step of generating the patient relationship network by mapping the sub-graph of doctor and patient according to the similarity health seeking behaviors between the patients further comprising:
generating patient relationship networks by mapping the sub-graph of doctor and patient according to the similarity health seeking behaviors between the patients;
obtaining a doctor cluster relationship by clustering the doctors involved in the patient relationship; and
generating the patient relationship network by connecting the patient relationship networks.

5. The claim settlement anti-fraud method of claim 1, wherein the step of computing a similarity between any two vertexes in the patient relationship network further comprising:
computing the similarity between any two vertexes in the patient relationship network according feature attributes and the health seeking behavior attributes of the corresponding patients, based on the similarity computation formula.

6. The claim settlement anti-fraud method of claim 5, wherein after the step of computing a similarity between any two vertexes in the patient relationship network further comprising:
updating a weight corresponding edge in the patient relationship network according to the similarity;
the step of computing an average similarity of each community close loop according to the similarity, which is implemented by the processor comprising:
computing the average similarity of each community close loop according to the updated weight in the patient relationship network, based on the average similarity computation formula.

7. The claim settlement anti-fraud method of claim 2, wherein the classification data comprises patient basic information, doctor basic information, and medical advice information.

8. (canceled)

9. A computer device, the computer device comprises a storage and a processor; wherein:
the storage, configured to storage computer programs;
the processor, configured to execute the computer programs to implement following steps:
generating a sub-graph of doctor and patient a sub-graph of doctor and medical advice according to medical data, and generating a fused large graph according to the sub-graph of doctor and patient and the sub-graph of doctor and medical advice, based on the graph computation technology;
generating a patient relationship network by mapping the sub-graph of doctor and patient according to the fused large graph; the patient relationship comprises a plurality of community close loops;
using a similarity computation formula, computing a similarity between any two vertexes in the patient relationship network according to feature parameters of the patients corresponding to the any two vertexes in the patient relationship network;
using an average similarity computation formula, computing an average similarity of each community close loop according to the similarity; and
confirming insurance fraud actions according to the average similarity;
wherein the similarity computation formula is:
$$sim 〈 A , B 〉 = \frac{{{\sum }^{n = 3}}_{i = 1} {A}_{i} {B}_{i}}{\sqrt{{{\sum }^{n = 3}}_{i = 1} {A}_{i}^{2}} \sqrt{{{\sum }^{n = 3}}_{i = 1} {B}_{i}^{2}}}$$
Wherein sim <A, B> represents the similarity, which is a cosine similarity, A and B represent the corresponding patients; A1 represents an age of the patient A, A2 represents a number of the medical advices of the patient A; A3 represents a medical cost of the patient A; B1 represents an age of the patient B; B2 represents a number of the medical advices of the patient B; A3 represents a medical cost of the patient B;
wherein the average similarity computation formula is:
$$ϕ ( P ) = \frac{{{\sum }^{n = N}}_{i = 1} {w}_{i}}{N}$$
wherein ϕ(P) represents the average similarity of the close loop of the community; P represents the community; N represents path coefficient of the community, and is a positive integer; W represents a weight of the path coefficient.

10. The computer device of claim 9, wherein the step of generating a sub-graph of doctor and patient a sub-graph of doctor and medical advice according to medical data, and generating a fused large graph according to the sub-graph of doctor and patient and the sub-graph of doctor and medical advice, based on the graph computation technology, which is implemented by the processor, further comprising:
classifying the acquired medical data to obtain classified data;
generating a classification relationship table according to association relationships of the classification data; the classification relationship table comprises a patient and doctor relationship table and a doctor and medical advice relationship table;
generating a bipartite graph according to the classification relationship table by a graph computation technology; and
generating the fused large graph according to the sub-graph of doctor and patient and the sub-graph of doctor and medical advice by a model combination technology of the graph computation technology.

11. The computer device of claim 9, wherein the step of generating a patient relationship network by mapping the sub-graph of doctor and patient according to the fused large graph, which is implemented by the processor, further comprising:
confirming similarity health seeking behaviors between the patients; and
generating the patient relationship network by mapping the sub-graph of doctor and patient according to the similarity health seeking behaviors between the patients.

12. The computer device of claim 11, wherein the step of generating the patient relationship network by mapping the sub-graph of doctor and patient according to the similarity health seeking behaviors between the patients, which is implemented by the processor, further comprising:
generating patient relationship networks by mapping the sub-graph of doctor and patient according to the similarity health seeking behaviors between the patients;
obtaining a doctor cluster relationship by clustering the doctors involved in the patient relationship; and
generating the patient relationship network by connecting the patient relationship networks.

13. The computer device of claim 9, wherein the step of computing a similarity between any two vertexes in the patient relationship network, which is implemented by the processor further comprising:
computing the similarity between any two vertexes in the patient relationship network according feature attributes and the health seeking behavior attributes of the corresponding patients, based on the similarity computation formula.

14. The computer device of claim 13, wherein after the step of computing a similarity between any two vertexes in the patient relationship network, which is implemented by the processor further comprising:
updating a weight corresponding edge in the patient relationship network according to the similarity;
the step of computing an average similarity of each community close loop according to the similarity, which is implemented by the processor comprising:
computing the average similarity of each community close loop according to the updated weight in the patient relationship network, based on the average similarity computation formula.

15. A computer readable storage medium, the computer readable storage medium stores a computer programs; the computer programs are executed by a processor for performing following steps:
generating a sub-graph of doctor and patient a sub-graph of doctor and medical advice according to medical data, and generating a fused large graph according to the sub-graph of doctor and patient and the sub-graph of doctor and medical advice, based on the graph computation technology;
generating a patient relationship network by mapping the sub-graph of doctor and patient according to the fused large graph; the patient relationship comprises a plurality of community close loops;
using a similarity computation formula, computing a similarity between any two vertexes in the patient relationship network according to feature parameters of the patients corresponding to the any two vertexes in the patient relationship network;
using an average similarity computation formula, computing an average similarity of each community close loop according to the similarity; and
confirming insurance fraud actions according to the average similarity;
wherein the similarity computation formula is:
$$sim 〈 A , B 〉 = \frac{{{\sum }^{n = 3}}_{i = 1} {A}_{i} {B}_{i}}{\sqrt{{{\sum }^{n = 3}}_{i = 1} {A}_{i}^{2}} \sqrt{{{\sum }^{n = 3}}_{i = 1} {B}_{i}^{2}}}$$
Wherein sim <A, B> represents the similarity, which is a cosine similarity, A and B represent the corresponding patients; A1 represents an age of the patient A, A2 represents a number of the medical advices of the patient A; A3 represents a medical cost of the patient A; B1 represents an age of the patient B; B2 represents a number of the medical advices of the patient B; A3 represents a medical cost of the patient B;
wherein the average similarity computation formula is:
$$ϕ ( P ) = \frac{{{\sum }^{n = N}}_{i = 1} {w}_{i}}{N}$$
wherein ϕ(P) represents the average similarity of the close loop of the community; P represents the community; N represents path coefficient of the community, and is a positive integer; W represents a weight of the path coefficient.

16. The computer readable storage medium of claim 15, wherein the step of generating a sub-graph of doctor and patient a sub-graph of doctor and medical advice according to medical data, and generating a fused large graph according to the sub-graph of doctor and patient and the sub-graph of doctor and medical advice, based on the graph computation technology, which is implemented by the processor, further comprising:
classifying the acquired medical data to obtain classified data;
generating a classification relationship table according to association relationships of the classification data; the classification relationship table comprises a patient and doctor relationship table and a doctor and medical advice relationship table;
generating a bipartite graph according to the classification relationship table by a graph computation technology; and
generating the fused large graph according to the sub-graph of doctor and patient and the sub-graph of doctor and medical advice by a model combination technology of the graph computation technology.

17. The computer readable storage medium of claim 15, wherein the step of generating a patient relationship network by mapping the sub-graph of doctor and patient according to the fused large graph, which is implemented by the processor, further comprising:
confirming similarity health seeking behaviors between the patients; and
generating the patient relationship network by mapping the sub-graph of doctor and patient according to the similarity health seeking behaviors between the patients.

18. The computer readable medium of claim 17, wherein the step of generating the patient relationship network by mapping the sub-graph of doctor and patient according to the similarity health seeking behaviors between the patients, which is implemented by the processor, further comprising:
generating patient relationship networks by mapping the sub-graph of doctor and patient according to the similarity health seeking behaviors between the patients;
obtaining a doctor cluster relationship by clustering the doctors involved in the patient relationship; and
generating the patient relationship network by connecting the patient relationship networks.

19. The computer readable medium of claim 15, wherein the step of computing a similarity between any two vertexes in the patient relationship network, which is implemented by the processor, further comprising:
computing the similarity between any two vertexes in the patient relationship network according feature attributes and the health seeking behavior attributes of the corresponding patients, based on the similarity computation formula.

20. The computer readable medium of claim 19, wherein after the step of computing a similarity between any two vertexes in the patient relationship network, which is implemented by the processor, further comprising:
updating a weight corresponding edge in the patient relationship network according to the similarity;
the step of computing an average similarity of each community close loop according to the similarity, which is implemented by the processor comprising:
computing the average similarity of each community close loop according to the updated weight in the patient relationship network, based on the average similarity computation formula.
